(19) United States
(12) Patent Application Publication
(10) Pub. No.: US 2026/0069784 A1
(43) Pub. Date: Mar. 12, 2026

(54) ATTACHABLE MONITOR FOR AUTO-INJECTOR DEVICES

(71) Applicant: Reciprocal Labs Corporation (dba Propeller Health), San Diego, CA (US)

(72) Inventor: Robert Louis BADDELEY, Madison, WI (US)

(73) Assignee: Reciprocal Labs Corporation (dba Propeller Health), San Diego, CA (US)

(21) Appl. No.: 19/107,551

(22) PCT Filed: Sep. 1, 2023
(86) PCT No.: PCT/US2023/031920

§ 371 Date: Feb. 27, 2025

Related U.S. Application Data
(60) Provisional application No. 63/374,405, filed on Sep. 2, 2022.

(51) Int. Cl. A61M 5/20

(52) U.S. Cl. CPC A61M 5/2033; A61M 2005/2006; A61M 2205/14; A61M 2205/3327; A61M 2205/3368; A61M 2205/584; A61M 2205/587

(57) ABSTRACT

An attachable adherence monitor for an auto-injector operable to deliver medication is disclosed. The adherence monitor includes a usage sensor that senses activation of the auto-injector to deliver the medication. The monitor includes a controller operable to determine activation of the auto-injector from the sensor. A storage device coupled to the controller stores the activation data.

### TECHNICAL FIELD

The present disclosure relates generally to a monitor for an auto-injector, and more specifically to an attachable adherence sensor for an automated injector.

### BACKGROUND

Injectable medications are becoming more prevalent. For example, one exciting new treatment in the medical field is the use of biologics for treatment of severe respiratory ailments specifically asthma in the respiratory space. A biologic is a pharmaceutical drug product manufactured in, extracted from, or semi-synthesized from biological sources. The term “biologic” is used herein to the refer to any biologic-based treatment or therapy, thereby encompassing any biologic medical product or pharmaceutical drug. The challenge with prescription of biologics is that they are still expensive (tens of thousands of dollars per dose) and require either self-administration or a visit to a care provider for the treatment to be administered. For example, Etrolizumab is a novel, dual-action anti-β7 integrin antibody for patients with inflammatory bowel diseases such as ulcerative colitis (UC) and Crohn's disease.

Another example is application of biologics for treating severe respiratory ailments. Respiratory ailments, such as asthma, remain a significant and costly public health problem. For example, in the United States, more than 22 million people have asthma. Worldwide, the World Health Organization estimates the population with asthma may be 235 million, and predicts that it will rise in the future. Similarly, recent studies by the Centers for Disease Control and Prevention have listed COPD as the third leading cause of death in the United States while estimating close to 15 million people may have COPD induced impaired lung function (Wheaton, A. G., et al., Employment and Activity Limitations Among Adults with Chronic Obstructive Pulmonary Disease-United States, 2013. MMWR Morb. Mortal Wkly. Rep., 27 Mar. 2015, 64(11), 289-294).

Many asthma exacerbations could be prevented with currently available treatments, however, only 1 in 5 asthmatics has the disease under control. Such treatments often rely on identifying a triggering condition of an asthma condition and properly administering treatment such as a medicament. One mechanism for a patient to self-administer a medicament is an inhaler. When a trigger event occurs, a patient may administer the medicament via a puff from the inhaler. There are rare instances where standard rescue inhaler medicaments may be ineffective for certain respiratory ailments and biologics provide effective treatment.

Biologics also require specific temperatures for storage and may only be applied shortly after storage as severe temperature changes destroy effectiveness of the biologic. Biologics are often injected via a mechanical auto-injector that may be operated by a user without assistance. Auto-injectors and pre-filled syringes are a class of medication devices that are intended to allow a patient to self-administer a medicament via needle into the body. In many cases these medicaments require strict adherence to temperature ranges. Thus medications must be warmed up to a certain temperature for injection for greater comfort to the patient as well as to allow the dispensing to happen more quickly due to the lower viscosity of the fluid. Finally, because of the high cost of these medications (many thousands of dollars each), there is a market for counterfeits, and there is a need to implement a mechanism to verify the authenticity of a given dose.

Auto-injectors are mechanical devices using spring force to automatically inject a needle for fluidly conveying the biologic to the user. Single-use prefilled auto-injectors have many potential advantages over the traditional pre-filled syringes with needle safety devices. For example, auto-injectors are designed to keep the needle out of sight of the user at all times during injection. Automatic injectors also offer increased convenience, ease of use, reduced risk of dosage error, and improved patient comfort. While such devices are designed for simple operation, the temperature as well as the actual operation of such devices is typically under the control of the patient. The expensive nature of certain medication such as biologics make it desirable to have monitoring of use of auto-injectors as, under certain circumstances, errors may be made by often untrained users thus potentially wasting expensive medication.

There is a need for a device that allows monitoring of the administration of biologics and other medication through an auto-injector. There is another need for a device that may detect the storage temperature of an auto-injector to insure that it is within parameters. There is another need for an adherence monitor that may be attached to existing auto-injectors.

### SUMMARY

One disclosed example is an attachable adherence monitor for an auto-injector operable to deliver medication. The monitor includes a usage sensor that senses activation of the auto-injector to deliver the medication. The monitor includes a controller operable to determine activation of the auto-injector from the sensor. A storage device is coupled to the controller to store the activation data.

In another implementation of the disclosed example adherence monitor, the medication is biologic-based. In another implementation, the example adherence monitor includes a temperature sensor. In another implementation, the controller is operable to periodically measure temperature of the medication from the temperature sensor. In another implementation, the controller stores the measured temperature in the storage device. In another implementation, the controller is operable to determine the medication is unsafe based on the measured temperature. In another implementation, the unsafe determination is made if the measured temperature is below a freezing temperature threshold or above an extreme temperature threshold. In another implementation, the adherence monitor includes a visual indicator. In another implementation, the controller is operable to activate the visual indicator to indicate the activation of the auto-injector. In another implementation, the visual indicator is a RGB LED. In another implementation, the controller is operable to control the RGB LED to emit a light of a first color if the medication is unsafe, emit a light of a second color if the medication requires an increase in temperature for administration, and a light of a third color if the medication is safe for administration. In another implementation, the adherence monitor includes a transceiver to transmit the activation data to an external device. In another implementation, the external device displays instructions relating to operating the auto-injector. In another implementation, the usage sensor is a limit switch that is triggered by contact with movement of the auto-injector when the medication is administered. In another implementation, the adherence monitor includes a cap detect sensor. The controller is operable to determine the auto-injector is ready for use based on an output of the cap detect sensor. In another implementation, the cap detect sensor is a limit switch, and the auto-injector includes a cap. The cap detect sensor senses when the cap is removed. In another implementation, the adherence monitor includes an accelerometer. The controller is operable to determine the auto-injector is ready for use based on an output of the accelerometer exceeding a threshold value. In another implementation, the adherence monitor includes a flexible circuit board mounting the controller and usage sensor. The flexible circuit board is wrapped around an internal component of the auto-injector. In another implementation, the adherence monitor includes a body holding the controller, usage sensor and storage device. The body is substituted for a component of the auto-injector.

Another disclosed example is a method of determining use of an auto-injector. An adherence monitor is attached to the auto-injector. The adherence monitor includes a controller and a use sensor. Activation of the auto-injector to deliver the medication is sensed via the use sensor. Activation of the auto-injector is determined from the sensor via the controller. The activation data is stored in a storage device.

In another implementation of the disclosed example method, the medication is biologic-based. In another implementation, the adherence monitor further includes a temperature sensor. In another implementation, the example method includes periodically measuring temperature of the medication from the temperature sensor. In another implementation, the example method includes storing the measured temperature in the storage device. In another implementation, the example method includes determining the medication is unsafe based on the measured temperature. In another implementation, the unsafe determination is made if the measured temperature is below a freezing temperature threshold or above an extreme temperature threshold. In another implementation, the adherence monitor includes a visual indicator. In another implementation, the example method includes activating the visual indicator to indicate the activation of the auto-injector. In another implementation, the visual indicator is a RGB LED. In another implementation, the example method includes controlling the RGB LED to emit a light of a first color if the medication is unsafe, emit a light of a second color if the medication requires an increase in temperature for administration, and a light of a third color if the medication is safe for administration. In another implementation, the adherence monitor includes a transceiver to transmit the activation data to an external device. In another implementation, the example method includes displaying instructions relating to operating the auto-injector on the external device. In another implementation, the usage sensor is a limit switch that is triggered by contact with movement of the auto-injector when the medication is administered. In another implementation, the example method includes determining the auto-injector is ready for use based on an output of a cap detect sensor. In another implementation, the cap detect sensor is a limit switch, and the auto-injector includes a cap. The cap detect sensor senses when the cap is removed. In another implementation, the example method includes determining the auto-injector is ready for use based on an output of an accelerometer exceeding a threshold value. In another implementation, the adherence monitor includes a flexible circuit board mounting the controller and usage sensor. The flexible circuit board is wrapped around an internal component of the auto-injector. In another implementation, the adherence monitor includes a body holding the controller, usage sensor and storage device. The body is substituted for a component of the auto-injector.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims.

### BRIEF DESCRIPTION OF THE DRAWINGS

The disclosure will be better understood from the following description of exemplary embodiments together with reference to the accompanying drawings, in which:

FIG. 1A shows an example prior art auto-injector;

FIG. 1B shows an example of another type of prior-art auto-injector;

FIG. 2A is a perspective view of an example attachable monitor for the auto-injector in FIG. 1A;

FIG. 2B shows a bottom cutaway view of the attachable monitor attached to one end of the auto-injector in FIG. 1A;

FIG. 2C shows a top perspective view of the attachable monitor with the cover removed;

FIG. 2D is a bottom perspective view of the example attachable monitor installed in the auto-injector in FIG. 1A;

FIG. 3A is a perspective view of another example attachable monitor that may be attached to the auto-injector in FIG. 2A;

FIG. 3B is a cutaway view of the attachable monitor in FIG. 3A installed on the auto-injector in FIG. 2A;

FIG. 3C is a perspective view of the attachable monitor in FIG. 3A installed on the auto-injector in FIG. 2A;

FIG. 4 is a block diagram of the electronic components of the attachable adherence monitors in FIG. 2A and FIG. 3A; and

FIG. 5 is a flow diagram of the routine for collecting monitoring data for the adherence monitors in FIG. 2A and FIG. 3A.

The present disclosure is susceptible to various modifications and alternative forms. Some representative embodiments have been shown by way of example in the drawings and will be described in detail herein. It should be understood, however, that the invention is not intended to be limited to the particular forms disclosed. Rather, the disclosure is to cover all modifications, equivalents, and alternatives falling within the spirit and scope of the invention as defined by the appended claims.

### DETAILED DESCRIPTION OF THE ILLUSTRATED EMBODIMENTS

The present inventions can be embodied in many different forms. Representative embodiments are shown in the drawings, and will herein be described in detail. The present disclosure is an example or illustration of the principles of the present disclosure, and is not intended to limit the broad aspects of the disclosure to the embodiments illustrated. To that extent, elements and limitations that are disclosed, for example, in the Abstract, Summary, and Detailed Description sections, but not explicitly set forth in the claims, should not be incorporated into the claims, singly or collectively, by implication, inference, or otherwise. For purposes of the present detailed description, unless specifically disclaimed, the singular includes the plural and vice versa; and the word “including” means “including without limitation.” Moreover, words of approximation, such as “about,” “almost,” “substantially,” “approximately,” and the like, can be used herein to mean “at,” “near,” or “nearly at,” or “within 3-5% of,” or “within acceptable manufacturing tolerances,”or any logical combination thereof, for example.

The present disclosure relates to an attachable sensor that collects data relating to status and determination of auto-injector use. The example sensor may be embedded inside the auto-injector or attached to the outside of the auto-injector. The attachable sensor may easily be mated to an existing auto-injector. The attachable sensor may be activated to sense use of the auto-injector and determine whether the conditions are optimal to inject the medication. The collected activation data may be transmitted to an external device via pairing. When the sensor is paired with a separate wirelessly connected external device, such as a smartphone or computer, additional information may be displayed. The external device may send data from the embedded sensor to a private network for collection and analysis.

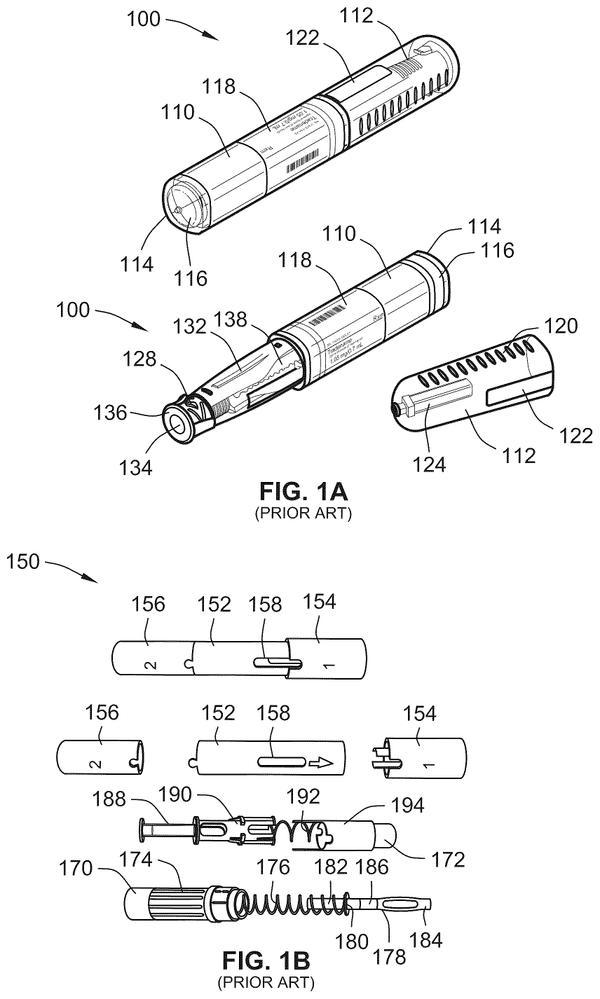

FIG. 1A shows front and rear perspective views of an example prior art auto-injector 100. The auto-injector 100 in this example is available from Genentech for medications such as Etrolizumab. The auto-injector 100 automatically inserts a needle in contact with skin, when activated. Prior to and during injection, the auto-injector keeps the needle out of view. Audio and visual features are included to signal the completion of the injection and to assist users with self-injection. The auto-injector 100 includes a main body 110 and a cap 112. The main body 110 includes a transparent rear end cover 114 that covers a spinning disk indicator 116. The main body 110 is generally cylindrical and includes an exterior label 118.

The cap 112 includes a series of air vents 120 and a viewing window 122. A needle sheath 124 extends from the interior closed end of the cap 112. The main body 110 holds a syringe (not shown) for the medication. The opposite end of the main body 110 from the rear end cover 114 holds a cylindrical needle assembly 128. The cylindrical needle assembly 128 holds a needle 130 that is attached to a spring propulsion mechanism 132. The end of the needle 130 extends through a cylindrical needle cover 134. The cylindrical needle cover 134 has an open aperture 136. A plunger rod 138 is propelled by the spring propulsion mechanism 132. A window 140 is provided along part of the length of the body 110. The plunger rod 138 is viewable through the window 140. When the cap 112 is inserted on the body 110, the needle sheath 124 is inserted in the aperture 136 of the needle cover 134 to protect the needle 130.

The auto-injector 100 is activated by removing the cap 112 from the body 110. The needle cover 134 is then lightly pressed perpendicularly onto the skin. Once activated, the auto-injector 100 automatically inserts the needle 130 via the spring mechanism 132 and dispenses the syringe contents. When injection is complete, the needle cover 134 extends and locks over the needle 130, keeping the needle out of view at all times during injection and protecting the user and others from accidental contact with the used needle. The auto-injector 100 also incorporates visual and auditory mechanisms designed to assist users with self-injection. For example, the spinning disk 116 is viewable through the rear end cover 114. The bottom of the spinning disk 116 has a shaft that mates with the auto-injector 100 allowing the spinning disk 116 to rotate. When the needle 130 is automatically inserted, the spinning disk 116 spins to indicate the drug administration is ongoing. In this example, the flat surface of the spinning disk 116 has a pattern of alternating blue and white colors to allow more visual indication of the spinning. An audible clicking sound is generated by the spring mechanism 132 to indicate whether drug administration is ongoing or completed. The spinning causes the flat surface colored blue and white on the spinning disk 116 at the end to spin. The spinning disk 116 also includes mechanisms to audibly click as the disk 116 spins to further indicate usage of the auto-injector 100. In this example, the spinning disk 116 spins roughly 7 times over the course of 5 seconds while the needle is inserted and the medication is injected. In addition, the plunger rod 138 moves across the viewing window 140 while the injection is in progress and thus provides another visible indicator of the administration of the medication.

FIG. 1B shows perspective and exploded views of another example prior art auto-injector 150, which is available from AbbVie for medication having the trade name Humira (adalimumab). The auto-injector 150 includes a cylindrical outer chassis 152 and two protective caps 154 and 156 that are attached on both ends of the outer chassis 152. The outer chassis 152 is cylindrical an includes a slot shaped window 158. The outer chassis 152 holds an activation button 170 on one end and a needle shroud 172 on the opposite end. The activation button 170 and needle shroud 172 are exposed when protective caps 154 and 156 are removed from the outer chassis 152.

The activation button 170 forms one end of a shuttle cylinder 174 that encloses a spring 176. The spring 176 is compressed by a plunger 178 prior to activation of the auto-injector 150. One end of the spring 176 rests on the activation button 170. The opposite end of the spring 176 is compressed against a stop member 180 of the plunger 178. The spring 176 is coiled around a stem 182 of the plunger 182. The plunger 178 includes an actuator 184 having a colored indicator area 186 that is visible through the window 158 in the outer chassis 152. A syringe 188 is held by a syringe holder 190. The syringe holder 190 is attached to a spring 192 and is inserted into a cylindrical inner chassis 194. The inner chassis 194 allows the movement of the syringe holder 190 and is enclosed by the needle shroud 172.

In operation, the auto-injector 150 is taken out of a refrigerator 15 to 30 minutes before injecting to allow the medicine in the syringe 188 to reach room temperature. After the caps 154 and 156 are removed, the needle shroud 172 is placed on the area of injection. Pressing the button 170 releases the spring 176 which provides spring force to push the actuator 184 of the plunger 178 into the syringe holder 190. The syringe holder 190 and needle of the syringe 188 are moved forward into the inner chassis 194. The needle is thus inserted into the injection area of the skin. The needle shroud 172 and compression of the spring 192 stops the syringe holder 190 and thus the needle after injection of the needle. The remaining spring force from the spring 176 moves a piston of the syringe 188 forward. The movement of the piston in the syringe 188 forces the medication through the needle to the injection area. The indicator area 186 may be seen through the window 158 in an end position near the needle shroud 172. A mechanical click sound is emitted via the contact of registration features of the syringe holder 190 with the interior of the outer chassis 152 to indicate the start of the injection.

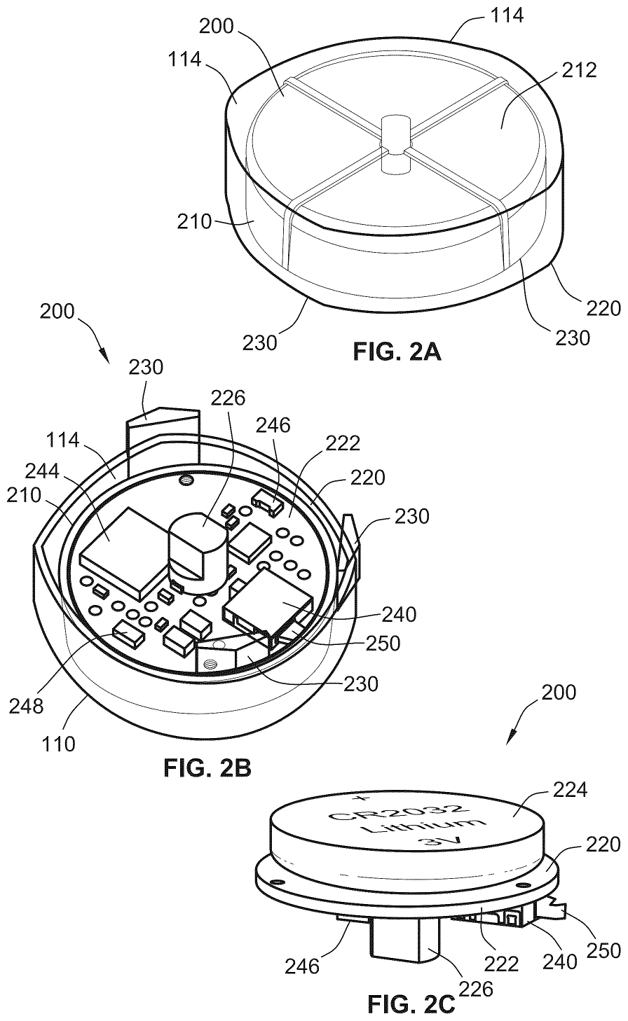

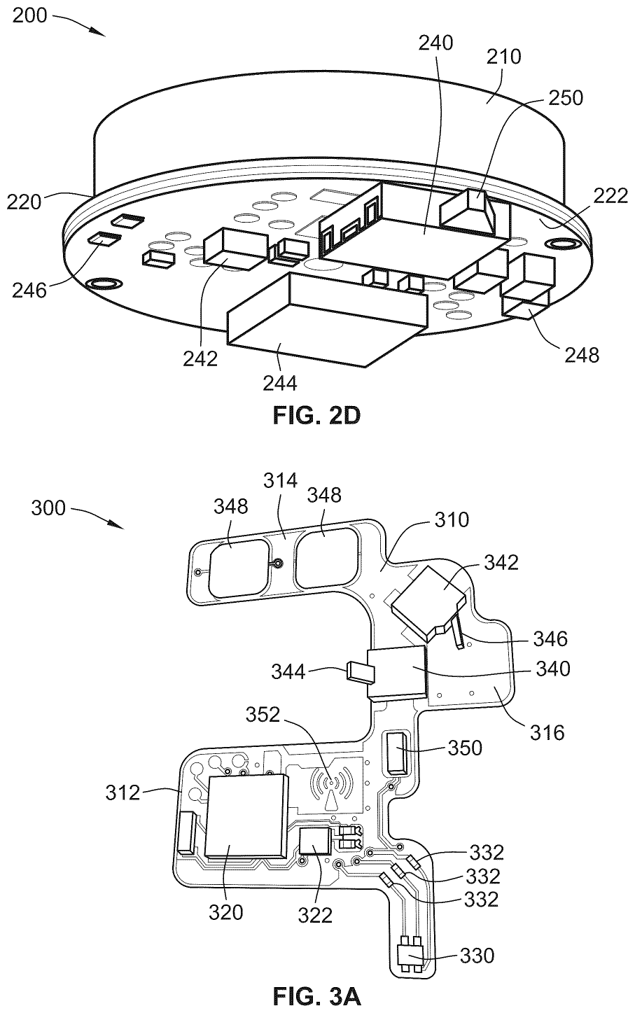

FIG. 2A shows a top perspective view of an attachable monitor 200 that replaces the spinning disk 116 enclosed by the rear end cover 114 of the auto-injector 100 in FIG. 1A. FIG. 2B shows a bottom cutaway view of the attachable monitor 200 attached to one end of the body 110 with the rear end cover 114 removed. FIG. 2C shows a top perspective view of the attachable monitor 200 with a cover removed. FIG. 2D shows a bottom perspective view of the attachable monitor 200 inserted in the rear end cover 114 of the auto-injector 100 in FIG. 1A.

As shown in FIG. 2A, the monitor 200 includes a cover 210 that is cylindrically shaped and approximately the same size as the spinning disk 116. The rear end cover 114 is transparent and is general cylindrical with an open end. When the monitor 200 is installed in the auto-injector 100, it replaces the spinning disk 116. Thus, the rear end cover 114 encloses the monitor 200 on one end of the main body 110 in FIG. 1A. Four tabs 230 extend from the open end of the rear end cover 114 that allow the rear end cover 114 to be attached to the end of the main body 110 of the auto-injector 100 in FIG. 1A.

The cover 210 may be painted with an exterior pattern 212 that is similar to that of the spinning disk 116 to provide a visual indication of the activation of the auto-injector 100. In this example, the pattern 212 is divided into quadrants of alternating blue and white colors. The cover 210 is mounted on a circular base 220 that holds a circular circuit board 222. A battery 224 is inserted between the circuit board 222 and the cover 210. The opposite side of the base 220 from the circuit board 222 supports a central shaft 226 that is similar to the shaft of the spinning disk 116 to allow attachment to an internal shaft in the auto-injector 100.

The monitor 200 is attached to the auto-injector 100 by removing the spinning disk 116 and inserting the shaft 226 into the internal rotating shaft of the auto-injector 100. In this example, the monitor 200 has the same diameter and general dimensions of the disk 116. The monitor 200 thus may drop-in replace the spinning disk 116. If necessary, the clear plastic rear end cover 114 can be replaced with a taller cover that maintains the same snap features as the original rear end cover 114, minimizing the impact to the assembly workflow of the auto-injector 100.

In this example, once attached to the auto-injector 100, the monitor 200 periodically measures the temperature of the medication in the auto-injector 100 and logs excursions of the auto-injector 100. The monitor 200 also indicates unsafe medication based on the measured temperature exceeding the safety temperature of the medication. The monitor 200 assumes the function of the spinning disk 116 in FIG. 1A to visually indicate operation of the auto-injector 100. Since the monitor 200 is inserted on an external shaft that rotates the needle, the inserted monitor 200 provides an indication that it is spinning to visually show the functioning of the auto-injector 100. Thus, the monitor 200, identical to the spinning disk 116, spins 7 rotations in about 5 seconds when the auto-injector 100 is activated.

The monitor 200 is powered by the battery 224 that is supported between the base 220 and the cover 210. Thus the battery 224 is on one side of the circuit board 222 and the sensor components are on the underside of the base 220. The battery may alternatively be placed on the spinning shaft under the base 220. Alternatively, a two part design may be employed where one part is the battery on a PCB with some spring contacts on the bottom and the second part is a PCB with a hole in the middle and two tracks for the spring to provide power.

With an accelerometer, the monitor 200 can detect motion to wake up and alert the patient using visual indicators, such as LEDs, about possible temperature excursions and spoilage, as well as indicate whether the unit has reached room temperature before the medication is taken. A mechanical limit switch is pressed repeatedly as the monitor 200 spins around inside the auto-injector. The sensor electronics of the monitor 200 may thus detect administration of the medication dose by the auto-injector 100 after a threshold number of switch presses is sensed.

In this example, the circuit board 222 mounts a limit switch 240, a sensor chip 242, a controller chip 244, and two RGB LEDs 246 and 248. The limit switch 240 includes a physical tab 250. The switch 240 is triggered by moving the tab 250. In this example, the tab 250 is moved when in contact with a matching tab on the interior of the main body 110 of the auto-injector 100. In this example, the RGB LEDs 246 and 248 may be illuminated red, yellow, or green color and may be viewed externally from the monitor 200.

The sensor chip 242 in this example is a LSM6DS3TR chip from STMicroelectronics and includes an accelerometer, temperature sensor, and gyroscope. The monitor 200 is initially in a sleep state when attached to the auto-injector 100. In the state, the monitor 200 is awoken periodically and takes temperature measurements and records the measurements in a log. The monitor 200 is woken up via an accelerometer interrupt triggered by the accelerometer on the sensor chip 242 that indicates the monitor 200 has been moved. The accelerometer interrupt provides a wake up for the electronic components on the circuit board 222. In this example, the temperature of the auto-injector 100 may be measured either from the temperature sensor on board the sensor chip 242 or a temperature sensor on board the controller 244. Alternatively, a separate temperature sensor chip may be mounted on the circuit board 222. In this example, on wake up, the controller 244 looks at the temperature history in the log to see if any excursions have occurred that render the medication unsafe. The controller 244 then lights the LEDs 246 and 248 green to indicate a safe condition. If an unsafe condition exists, the controller 244 lights the LEDs 246 and 248 red. If the medication needs to be warmed to room temperature for administration, the controller 244 lights the LEDs 246 and 248 yellow.

Alternatively, wake up of the electronic components may be via a Hall Effect sensor. For this method, a magnet is attached on the body of the auto-injector 100 or the clear rear end cap 114. The Hall effect sensor would be triggered when the magnetic value changes indicating the auto-injector 100 has been activated.

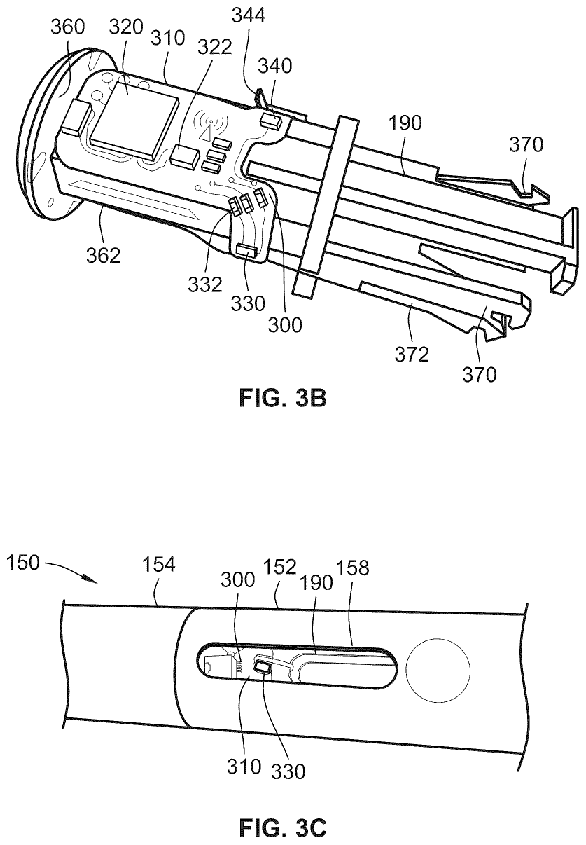

FIG. 3A shows a perspective view of another type of attachable adherence monitor 300 for the auto-injector 150 shown in FIG. 1B. FIG. 3B shows a perspective view of the adherence monitor 300 attached to syringe holder 190. FIG. 3C shows the outer chassis 152 of the auto-injector 150 with the adherence monitor 300 installed. As shown in FIGS. 3B-3C, the adherence monitor 300 may be wrapped around the syringe holder 190 and thus moves with the syringe holder 190 when the auto-injector 150 is activated. As will be explained the adherence monitor 300 is attachable to the syringe holder 190 and is visible through the window 158 of the outer chassis 152. The adherence monitor 300 is designed to be assembled into the auto-injector 150 as an additional step in its assembly, or snapped onto the pre-filled syringe. Once the battery is inserted, the adherence monitor 300 becomes functional.

As shown in FIG. 3A, the adherence monitor 300 includes a flexible circuit board 310 that wraps around a component inside the medication delivery device inside, containing all of the components, and positioned in such a way that it does not interfere with the operation of the injector. The flexible circuit board 310 has a main section 312 and battery contact section 314 parallel to the main section 312. A lateral section 316 connects the main section 312 with the battery contact section 314. The main section 312 includes a microprocessor chip 320 and an accelerometer chip 322. The accelerometer chip 322 in this example includes a temperature sensor and the accelerometer.

The main section 314 includes an RGB LED 330 with three inputs for the three colors. Each of the inputs are controlled via signals from the microprocessor chip 320 that are sent through a resistor 332 to the LED 330. A first limit switch 340 and a second limit switch 342 are mounted on the circuit board 310 on the lateral section 316. The limit switch 340 is triggered by moving a mechanical tab 344. The second limit switch 342 is triggered by moving a corresponding mechanical tab 346. In this example, the limit switch 340 serves as a usage sensor indicating activation of the auto-injector 150, while the limits switch 342 serves as a cap detection sensor indicating whether the cap 154 is removed from the auto-injector 150. The battery contact section 314 includes two electrodes 348 that allow connection to a battery to power the components of the circuit board 310. A smoothing capacitor 350 is provided for smoothing the power from the battery.

In this example, the microprocessor chip 320 includes internal memory that is used to store a temperature log as well as data indicating activation of the auto-injector. The microprocessor chip 320 also includes an internal radio. In this example, the internal radio is a BLE transceiver. The circuit board 310 includes an on-board antenna 352 for transmitting and receiving BLE signals.

As shown in FIGS. 3B-3C, the circuit board 310 has a shape to make the RGB LED 330 visible through the window 158. The syringe holder 190 includes a circular back plate 360 that includes an aperture that allows the syringe 188 to be extended in a cocked position as shown in FIG. 1B. The circular back plate 360 is joined to a cylindrical body 362 that holds the syringe 188. The circuit board 310 is fabricated from a flexible material and thus may be wrapped around the exterior of the cylindrical body 362 and attached with adhesive tape. Alternatively, registration features may be provided in the circuit board 310 to snap into corresponding features on the syringe holder 190. The syringe holder 190 includes a series of lateral hooks 370 that contact features on the interior surface of the outer chassis 152 in FIG. 1B. The lateral hooks 370 create a sound when pushed into the outer chassis 152. A slot 372 is provided that allows the syringe to be viewed from the window 158.

The microprocessor 320 detects the presence of the cap 154 through the cap detection limit switch 342. The activation of the auto-injector 150 may be detected because the movement of the syringe holder 190 from releasing the spring 176 will trigger the tab 344 of the limit switch 340. The temperature of the medication in the auto-injector 150 is monitored by the microprocessor 320 through either an internal temperature sensor or the temperature sensor on the accelerometer chip 322. The microprocessor 320 may cause red, green or yellow light to be emitted from the LED 330 depending on the status of the auto-injector 100 from the recorded temperatures. Alternatively, the adherence monitor 300 may also be a rigid board inside an enclosure that attaches to the auto-injector 150.

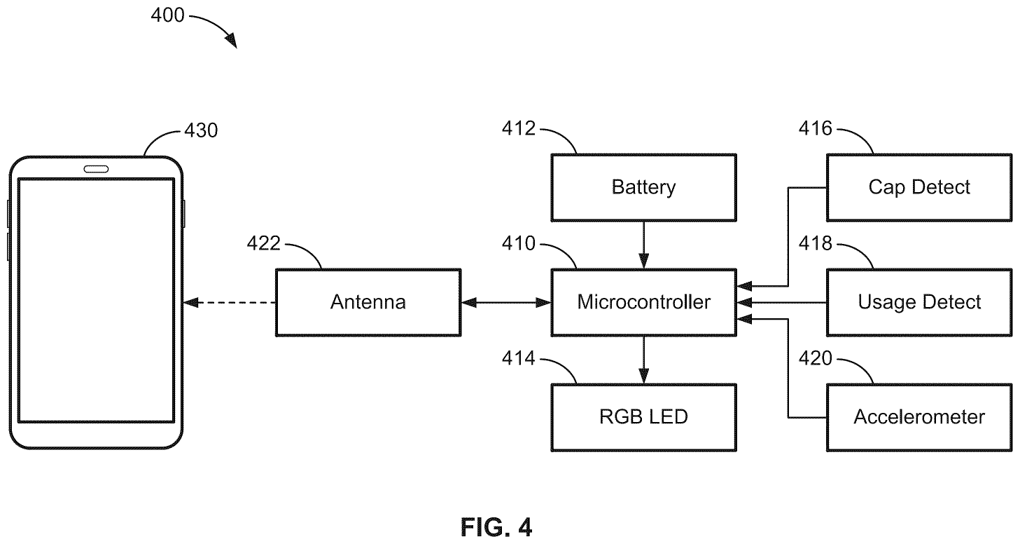

FIG. 4 is a block diagram of the sensor electronics 400 in both the monitor 200 and 300. Although, the monitors 200 and 300 include features for specific types of auto-injectors, it is to be understood that the sensor electronics 400 may be adapted with any type of auto-injector. The sensor electronics 400 consists of a microcontroller 410, a battery 412, an RGB LED 414, a cap detect limit switch 416, a usage detect limit switch 418, and an accelerometer 420. The microcontroller 410 is responsible for monitoring the temperature of the auto-injector, the inputs from the limit switches 416 and 418, the outputs to the LED 414, storing data, running a clock, and performing Bluetooth communication of stored data. The battery 412 is the power supply for the unit. In this example, the microcontroller 410 includes an on die temperature sensor, a radio such as a BLE transceiver, and a memory to store collected data. In this example, an on-board antenna 422 may transmit BLE signals to an external device 4300.

The RGB LED 414 may be controlled to output green, red, and yellow light to indicate different operational status indicators of the monitor and information relating to the auto-injector. Alternatively, separately colored LEDs may be used for each color instead of a single LED. The cap detect limit switch 416 detects whether the cap is on or off. When a cap is off, the limit switch 416 provides a signal that the microcontroller 410 will use to indicate that the auto-injector is ready to use. For example, the LED 414 may be controlled to emit green light to indicate that the injector is ready for use once the cap is removed.

The usage detect limit switch 418 detects whether the medication has been dispensed based on the triggering of mechanical parts of the auto-injector. Other detection methods, including but not limited to Hall effect sensors, IR sensors, or microphones could be used in place of either limit switch. The accelerometer 420 is an optional component that detects the orientation of the auto-injector. The orientation of the auto-injector may thus indicate that the auto-injector is readied for use. The sensor electronics may have a separate temperature sensor that can be used in addition to or instead of the die temperature sensor on the microcontroller 410 or the die temperature sensor on the accelerometer chip.

During the assembly process, the MAC address of the microcontroller 410 is noted or programmed. In addition, a randomly generated 32 byte key is created and stored on the unit and retained in a log stored on the internal memory of the microcontroller 410. This key is then transferred to a secure location on a private network, where it can be used to authenticate the sensor electronics in the future. Additionally, an expiration date can be programmed into the sensor.

Once the auto-injector is assembled, the sensor electronics 400 are placed in a sleep state either by reaching a threshold temperature or by reaching a predetermined period of time, or through an external command triggered wirelessly. In this sleep state, the microcontroller 410 is programmed to wake at regular intervals to measure the temperature. For example, a regular interval may be selected between 30 minutes and four hours based on a desired balance of preserving battery life and ability to accurately detect excursions. In this example, the microcontroller 410 stores the measured temperature at daily intervals, as well as more frequently during larger temperature changes. An internal memory on the microcontroller 410 stores the status of the medication as well, noting whether it has been spoiled (based on a higher temperature) and for what reason.

There are multiple ways a dose can be considered spoiled. First, if the dose has been below a freezing temperature threshold for longer than a given period of time. Second, if the dose has been above an extreme temperature threshold for longer than a given period of time. Third, if the dose has been above a refrigerated temperature threshold (but below the extreme temperature threshold) longer than a given period of time that may be longer than the extreme period. Finally, the dose may be considered spoiled if the programmed expiration period of the dose has elapsed.

During the sleep period of the sensor, the sensor electronics 400 maintain a low power state and do not display any status on the LED 414. The microcontroller 410 may advertise its status over the Bluetooth radio at infrequent intervals to conserve energy. Alternatively, during the sleep period, the radio may be disabled entirely.

There are two ways to wake up the sensor electronics 400: either a physical motion that presses or releases the cap limit switch 416 (such as removal of the cap), or motion detected by the accelerometer 420 beyond a threshold. In either case, this motion triggers the sensor electronics 400 to enter into an awakened state.

In the awakened state, the sensor electronics 400 determine the status of the auto-injector and lights different colors accordingly on the LED 414. The routine on the microcontroller 410 will light the LED 414 red to indicate the dose is spoiled or expired or otherwise not fit for use. The routine on the microcontroller 410 will light the LED 414 yellow to indicate the dose is valid, but its temperature is too cold to be injected immediately. The microcontroller 410 will light the LED 414 green to indicate the dose is valid, and its temperature is warm enough to be injected. If the LED 414 is not lit because the battery has depleted, the LED 414 will not turn on, and the dose should be assumed to be expired or spoiled.

Additionally, while in this awakened state, the sensor electronics 400 may seek connection to an external device 430 via the wireless Bluetooth radio on the microcontroller 410. The microcontroller 410 will advertise its presence at an increased rate to ease connection speed. Once an external device such as a smartphone, a gateway, a computer, has connected to the microcontroller 410 a variety of functions may be performed. The functions include querying the private network for the authentication key for that monitor and use the authentication to validate the authenticity of the monitor through a special handshaking process. Another function is a download of log entries of past events, including temperature records. Another function, is receiving frequent notifications about the current status of the monitor, including whether the dose is expired or spoiled, the current temperature of the auto-injector, the current orientation of the auto-injector, whether the cap is on or off, and whether the dose has been administered.

In this example, the microcontroller 410, employs a wireless communication protocol for the built in radio such as the Bluetooth Low Energy (BTLE) protocol. BTLE is a short-ranged, low-powered, protocol standard that transmits data wirelessly over radio links in short range wireless networks. After the microcontroller 410 and external device 4300 have been paired with each other using a BTLE passkey, the microcontroller 410 automatically synchronizes and communicates information relating to medicament device usage with the external device 430. In other implementations, other types of wireless connections are used (e.g., infrared or IEEE 802.11).

The monitor may also be configured to communicate more directly with the external device 430. For example, if a network adapter of the monitor is configured to communicate via a wireless standard such as IEEE 802.11 or LTE, the adapter may exchange data with a wireless access point such as a wireless router, which may in turn communicate with the external device 430. These two methods of communicating are not mutually exclusive, and the monitor may be configured to communicate with both the external device 430 and an application server, for example using redundant transmission to ensure event data arrives at the application server or to provide information directly to the external device 430 while the application server is determining what notification to provide in response to an event.

By collecting this information, the status of the auto-injector in more detail than is afforded with the LED 414 may be displayed to the user on the external device. The external device may also execute an application to use the data received from the monitor to guide the patient through administration of the dose, with feedback and instructions given as the patient is performing the steps.

Once the patient presses the button on the medication device to release the medicament, the monitor detects the usage through the limit switch 418 and initiates a timer. During this period, which is typically 10 seconds, the LED 414 is controlled to blink a blue color output, and afterwards the LED 414 turns off. The purpose of the blinking blue light is to aid the patient in ensuring they do not remove the needle until the full dose has been released into the skin.

After the dose has been administered, the monitor generates another event that is stored in the memory of the microcontroller 410 indicating the usage and the timestamp of the usage. This event is then transmitted to the external device and then to the private storage.

In some situations, the patient may not connect their monitor wirelessly prior to using the auto-injector. In this case, the patient cannot verify the authenticity of the dose. However, with the use of the LED 414, the patient can be sure that the dose is not spoiled or expired, and that it is at a comfortable temperature for injection. After the dose has been administered, the monitor will continue to advertise its availability so that once the monitor is within range and able to connect to a compatible Bluetooth device, the temperature history and event history may be downloaded. Thus, it is not necessary for the monitor to be connected in order to be useful, and the data can still be synced eventually when convenient.

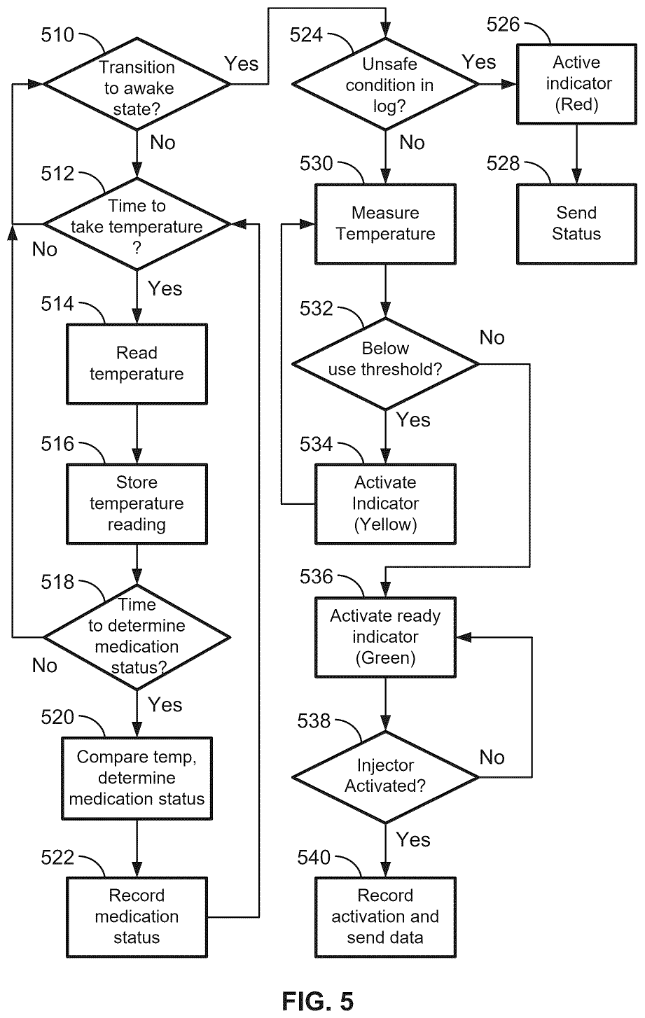

FIG. 5 is a flow diagram of a data collection routine to collect operational data for the use of an auto-injector, according to certain aspects of the present disclosure. The flow diagram in FIG. 5 is representative of example machine readable instructions for the process of collecting data from the use of an auto-injector. In this example, the machine-readable instructions comprise an algorithm for execution by: (a) a processor; (b) a controller; and/or (c) one or more other suitable processing device(s). The algorithm may be embodied in software stored on tangible media such as flash memory, CD-ROM, floppy disk, hard drive, digital video (versatile) disk (DVD), or other memory devices. However, persons of ordinary skill in the art will readily appreciate that the entire algorithm and/or parts thereof can alternatively be executed by a device other than a processor and/or embodied in firmware or dedicated hardware in a well-known manner (e.g., it may be implemented by an application specific integrated circuit [ASIC], a programmable logic device [PLD], a field programmable logic device [FPLD], a field programmable gate array [FPGA], discrete logic, etc.). For example, any or all of the components of the interfaces can be implemented by software, hardware, and/or firmware. Also, some or all of the machine-readable instructions represented by the flowcharts may be implemented manually. Further, although the example algorithm is described with reference to the flowchart illustrated in FIG. 5, persons of ordinary skill in the art will readily appreciate that many other methods of implementing the example machine readable instructions may alternatively be used. For example, the order of execution of the blocks may be changed, and/or some of the blocks described may be changed, eliminated, or combined.

The routine first determines whether to transition from the sleep state to an awake state by checking the accelerometer reading to see if the auto-injector is about to be used (510). Alternatively, the awake state may be determined by the cap limit switch being triggered by removal of the protective cap. If there is no transition, the routine determines whether a temperature reading is taken (512). If a temperature reading should be taken, the routine activates the controller to take a reading (514). The reading is then stored in the log in memory (516). The routine then determines whether a medication status should be determined (518). If the medication status should be determined, the routine determines whether the medication is safe by comparing the logged temperature to a freeze threshold and an extreme threshold (520). The status of the medication is then recorded in the memory (522).

If the routine transitions to the awake state, the routine checks the temperature log to determine if any unsafe conditions are recorded (524). If an unsafe condition is recorded, the routine will activate a visual indicator such as a LED to emit a certain color such as red (526). The routine will then activate the radio and transmit the status of the auto-injector and the temperature log to an external device (528)

If no unsafe condition is recorded, the routine will measure the temperature of the medication (530). The routine will determine whether the temperature is below a dosing threshold (532). If the temperature of the medication is below the dosing threshold such as room temperature, the routine will activate the visual indicator to emit a certain color such as yellow (534). The routine will then loop back to continue to measure the temperature (530). If the medication is above the dosing threshold, the routine will activate the visual indicator to emit a certain color such as green (536). The routine will determine whether the auto-injector has been activated to deliver the medication through determining whether the usage sensor such as a limit switch has been triggered (538). If the auto-injector is activated, the routine will assign a time stamp and send the activation data to an external device (540). If the auto-injector is not activated, the routine will loop back and wait for activation (538).

As used in this application, the terms “component,” “module,” “system,” or the like, generally refer to a computer-related entity, either hardware (e.g., a circuit), a combination of hardware and software, software, or an entity related to an operational machine with one or more specific functionalities. For example, a component may be, but is not limited to being, a process running on a processor (e.g., digital signal processor), a processor, an object, an executable, a thread of execution, a program, and/or a computer. By way of illustration, both an application running on a controller, as well as the controller, can be a component. One or more components may reside within a process and/or thread of execution, and a component may be localized on one computer and/or distributed between two or more computers. Further, a “device” can come in the form of specially designed hardware; generalized hardware made specialized by the execution of software thereon that enables the hardware to perform specific function; software stored on a computer-readable medium; or a combination thereof.

The terminology used herein is for the purpose of describing particular embodiments only, and is not intended to be limiting of the invention. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art. Furthermore, terms, such as those defined in commonly used dictionaries, should be interpreted as having a meaning that is consistent with their meaning in the context of the relevant art, and will not be interpreted in an idealized or overly formal sense unless expressly so defined herein.

While various embodiments of the present invention have been described above, it should be understood that they have been presented by way of example only, and not limitation. Although the invention has been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application. Thus, the breadth and scope of the present invention should not be limited by any of the above-described embodiments. Rather, the scope of the invention should be defined in accordance with the following claims and their equivalents.

What is claimed is:

1. An attachable adherence monitor for an auto-injector operable to deliver medication, comprising:
a usage sensor that senses activation of the auto-injector to deliver the medication;
a visual indicator;
a controller operable to determine activation of the auto-injector from the sensor, and to activate a visual indicator to indicate the activation of the auto-injector; and
a storage device coupled to the controller to store activation data.

2. The adherence monitor of claim 1, further comprising a temperature sensor wherein the controller is operable to periodically measure temperature of the medication from the temperature sensor and determine the medication is unsafe based on the measured temperature, wherein an unsafe determination comprises the measured temperature being below a freezing temperature threshold or above an extreme temperature threshold.

3. The adherence monitor of claim 1, wherein the controller is operable to control the visual indicator to emit a light of a first color if the medication is unsafe, emit a light of a second color if the medication requires an increase in temperature for administration, and a light of a third color if the medication is safe for administration.

4. The adherence monitor of claim 1, further comprising a transceiver to transmit the activation data to an external device, wherein the external device displays instructions relating to operating the auto-injector.

5. The adherence monitor of claim 1, wherein the usage sensor is a limit switch that is triggered by contact with movement of the auto-injector when the medication is administered.

6. The adherence monitor of claim 1, further comprising a cap detect sensor, wherein the controller is operable to determine the auto-injector is ready for use based on an output of the cap detect sensor.

7. The adherence monitor of claim 6, wherein the cap detect sensor is a limit switch, and wherein the auto-injector includes a cap, wherein the cap detect sensor senses when the cap is removed.

8. The adherence monitor of claim 1, further comprising an accelerometer, wherein the controller is operable to determine the auto-injector is ready for use based on an output of the accelerometer exceeding a threshold value.

9. The adherence monitor of claim 1, further comprising a flexible circuit board mounting the controller and usage sensor, wherein the flexible circuit board is wrapped around an internal component of the auto-injector.

10. A method of determining use of an auto-injector, the method comprising:
attaching an adherence monitor to the auto-injector, the adherence monitor including a controller and a use sensor, a visual indicator;
sensing activation of the auto-injector to deliver medication via the use sensor;
determining activation of the auto-injector from the sensor via the controller;
activating the visual indicator to indicate the activation of the auto-injector; and
storing activation data in a storage device.

11. The method of claim 10, wherein the adherence monitor further comprises a temperature sensor, and the method further comprises periodically measuring temperature of the medication from the temperature sensor, and determining the medication is unsafe based on the measured temperature, wherein an unsafe determination comprises the measured temperature being below a freezing temperature threshold or above an extreme temperature threshold.

12. The method of claim 10, further comprising controlling the visual indicator to emit a light of a first color if the medication is unsafe, emit a light of a second color if the medication requires an increase in temperature for administration, and a light of a third color if the medication is safe for administration.

13. The method of claim 10, wherein the adherence monitor includes a transceiver to transmit the activation data to an external device.

14. The method of claim 13, further comprising displaying instructions relating to operating the auto-injector on the external device.

15. The method of claim 10, wherein the usage sensor is a limit switch that is triggered by contact with movement of the auto-injector when the medication is administered.

16. The method of claim 10, further comprising determining the auto-injector is ready for use based on an output of a cap detect sensor.

17. The method of claim 16, wherein the cap detect sensor is a limit switch, and wherein the auto-injector includes a cap, wherein the cap detect sensor senses when the cap is removed.

18. The method of claim 10, further comprising determining the auto-injector is ready for use based on an output of an accelerometer exceeding a threshold value.

19. The method of claim 10, wherein the adherence monitor includes a flexible circuit board mounting the controller and usage sensor, wherein the flexible circuit board is wrapped around an internal component of the auto-injector.

20. The method of claim 10, wherein the adherence monitor includes a body holding the controller, usage sensor and storage device, wherein the body is substituted for a component of the auto-injector.
